(19) United States
(12) Patent Application Publication
(10) Pub. No.: US 2008/0253916 A1
(43) Pub. Date: Oct. 16, 2008

(54) Methods of Making Stents

(75) Inventors: Naim Istephanous, Roseville, MN (US); Keith Hanover, Kenwood, CA (US); Darrel F. Untereker, Oak Grove, MN (US)

Correspondence Address: MEDTRONIC, INC., 710 MEDTRONIC PARKWAY NE, MINNEAPOLIS, MN, 55432-9924, US

(73) Assignee: Medtronic, Inc.

(21) Appl. No.: 12/045,731

(22) Filed: Mar. 11, 2008

Related U.S. Application Data
(63) Continuation of application No. 10/656,730, filed on Sep. 5, 2003.

(51) Int. Cl. A61F 2/06

(52) U.S. Cl. 419/5

(57) ABSTRACT

Manufacturing methods are provided to build modulated medical devices and segments of the devices for applications in the field of intraluminal intervention, reconstruction, or therapy. The methods, comprise steps of metal injection molding and processes of modulation, improve the manufacturability of the devices and/or expand the design alternatives for the devices. The modulated medical devices and their segments, made from the present method inventions, enhance the versatility in intraluminal treatments.

This application is a continuation application of, and claims priority to, co-pending U.S. patent application Ser. No. 10/656,730, to Istephanous, et. al, filed Sep. 5, 2003, which is hereby incorporated by reference in its entirety. The invention relates to modulated stents and methods of making the stents. The segments of the stents are made by metal injection molding process that increases the versatility in stent design, allows the capability in stent modulation, and reduces the commonly encountered variations in the conventional manufacturing processes of the stents.

### FIELD OF THE INVENTION

### Background of the Invention

There are various tubular or lumen structures (collectively “lumen(s)”) in the body of human or other animals. Examples of such lumens are: vascular and neurovasular vessels, bronchi, bile duct, liver ducts, pancreatic duct, stomach, esophagus, colons, ileum, jejunum, rectum, urinary tract, ear canals and ducts, lacrimal ducts, nasolacrimal ducts, sinus. Those lumens are functioned to store or transport nutrient and waste between organs or to and from outside the body. Non-restricted flow of nutrient or waste inside the lumens is essential in maintaining the health of a body.

Aging, life-style (e.g., eating habit, exercise routine, living and working environments), diseases (e.g., malignant tumor, stenosis), injury, surgery, or generic effects could cause blockage, occlusion, narrowing, or collapse (collectively “blockage”) of the lumens, thus diminish their functions in sustaining life. Endo-structural stenting is a well-recognized procedure, sometimes in conjunction with other surgical or non-surgical procedures (e.g., ablation, balloon dilation, laser treatment, or atherectomy), to repair the blockages.

In endo-structural stenting, an unexpanded or compressed stent (partly for the reason of ease of delivering the stent to the treatment site) is delivered, expanded, and affixed at the site of blockage to maintain a pathway for nutrient or waste. In order to serve well the above-mentioned functions, a stent is designed generally with the following considerations: ease of deployment through the tortuous pathways (e.g., having optimal flexibility and distinct radiopacity in the stent structure), in compliance with the deployment tools such as balloon catheters (e.g., self-expandable or minimum force required to transform from the unexpanded configuration to the expanded configuration), capability of maintaining the expanded configuration (i.e., low or no recoiling) to withstand radial compression force from the lumen, capability of providing adequate flow capacity throughout the service life of the stent (e.g., preventing the restenosis), capability of avoiding or easing the invasive effects to the lumens, and capability of providing other therapeutic treatments when needed.

Stents can be made from biocompatible metals or non-metals. A number of patents or applications have been issued or published pertaining various metal stents and methods of making the metal stents.

U.S. Pat. No. 4,655,771 issued to Wallsten discloses a stent formed from a thread wire. The stent is deployed in a contracted form and later self-expands when released in the blood vessel.

U.S. Pat. No. 5,628,787 issued to Mayer discloses a clad composite stent formed of multiple filaments arranged in a braided configuration. Each filament has a central core and a case surrounding the core.

U.S. Pat. No. 5,651,174 issued to Schwartz et al. discloses a method for making a stent by providing a flat wire band formed into a zigzag pattern, applying a polymeric film to the flat wire band, and bending the band and polymeric film into a cylindrical shape.

U.S. Pat. No. 5,984,963 issued to Ryan et al. describes endovascular stents being cut from a flat sheet of material. The stents also have latching mechanisms that do not protrude significantly into the lumen of the stent and do not significantly increase the bulk of the stent.

U.S. Pat. No. 6,193,829 issued to Acciai et al. and U.S. Pat. Application US2001/0012960 A1 published for the same inventors describe a stent jointed by two filaments. Laser welding or injection molding of a joint material are used to joint the filaments. Related methods and tooling for forming a stent are also disclosed.

U.S. Pat. No. 6,206,915 issued to Fagan et al. describes a stent comprising inner lumen and outer lumen, and at least one protrusion provided on at least one of the inner and outer members and extending across the space so as to cause a friction fit between the inner and outer lumens. The stent also includes a pattern of perforation across both the inner and outer members to permit the stent to expand radially.

U.S. Pat. Application US 2002/0138131 published for Solovay et al. describes a stent with a plurality of support elements. The stent includes first and second terminal ends and a length extending between the terminal ends.

European Pat. No. EP 1,208,814 issued to McGuinness discloses a stent manufactured from metal tubing, having a hollow cylindrical body made with a plurality of rings. The rings each extend circumferentially around the cylindrical body and include an undulating series of angulated peaks and valleys.

WIPO Pat Application WO 00/54704 published for Jalisi discloses a composite stent having a substrate tube placed within a metal cladding tube. The laminate tube then undergoes a series of rolling or cold-drawing processes interspersed with heat-treating to release built up stresses. The finished laminate tube is then cut or etched to form a stent pattern.

The metal stents described in the above patents and applications are generally in tubular or similar configurations and conventionally made from thin sheet metals, wires, or tubes. More specifically, their structures are typically formed with repetitive segments, namely crowns or hoops, i.e., each crown or hoop has same or similar design patterns. And the crowns or hoops are constructed with a network of rings, which are conventionally made from metal wires, tubes, or sheet stocks.

Manufactures of the tubular stents from wires, tubes, or sheet stocks are tedious and often involving multiple secondary operations. Such as, in an initial step, multiple thin sections (i.e., generally a few thousandth of an inch in diameter or in thickness) are cut from a metal tube or sheet stock, or formed and welded from a metal wire. Then, predetermined sinusoidal patterns are formed, usually by bending, from the thin sections of tubes or wires. The sinusoidal parts are then spot welded at various joints to form a network of crowns. Depending on the length requirement, several tubular crowns are then welded together at various joints to form a stent. In addition, associated operations such as aligning, tumbling, annealing, polishing, or straightening are often incorporated to achieve the predetermined patterns and specified mechanical requirements. The sizes of the crowns are conceivable small as they are constrained by the inner diameter of the treated lumens (e.g., coronary or carotid vessel). Furthermore, there are constant demands in reducing metal-to-artery ratio and strut thickness to improve the maneuverability and performance of the stent in small vessels. As a result, handling and aligning such small crowns and thin struts are known to be inherent hurdle in the manufacturing of the stents. Occurrences of manufacturing variations (e.g., mis-alignment of the joints between the thin sections, weakened joints as a result of laser or annealing operation, altered mechanical property or integrity from polishing, tumbling or annealing, undetected and undesired residue from various operation steps) are equally burdensome to the stent manufacturers. Consequently, the costs incurred from the efforts to reduce the variations and to improve the handling in manufacturing are often accounted for a significant portion of the overall stent cost. Costly capital equipment and disposable tooling are often accounted for a significant portion of expenditure to improve throughput and production yields. Therefore, there are needs for alternative manufacturing methods to improve the handling and to reduce the variations in stent manufacturing, and ultimately to lower the overall stent costs.

The conventional stent manufacturing methods seemingly also have hindered the innovation of stent design. More noticeable, the choices of stent material are limited to the groups of metals that are suitable for the forming processes of wires, sheets, or tubes. The cold works in the wire drawing or tube/sheet forming process can further adversely affect the properties of the materials in the already limited pool of choice. In effect, the processes of wire, sheet, or tube have restricted the feature that a stent may be designed. For example, U.S. Pat. No. 6,503,271 issued to Duerig et al. describes feature restrictions that stent design has to follow in order to reduce or prevent twist or whip. Less apparent, innovations in stent design (e.g., drug-storing reservoirs, fastening pads, interlocking pads) seemingly have not been nearly explored in the field of using metal wires, sheets, or tubes as the starting materials. Stent designers appear to have no choice but to shelve their innovated ideas due to lack of feasible or cost effective manufacturing techniques. Therefore, synchronization between stent manufacturing and design (e.g., removing the commonly encountered restrictions and/or allowing flexibility in stent designs) not only can fulfill a long felt or nagging need but also most likely to have long-lasting boosting effects to the stent industry. It is foreseeable that innovation in stent application likely will excel when the paradigm of using metal wires, sheets, or tubes is overcome.

The stents are typically delivered to the treatment sites by a catheter or an equivalent delivery system. The operating physician often relies on a diagnostic imaging technology (e.g., x-ray, fluoroscope, CT scan, MRI) to maneuver, position, and affix the stent to the implantation site. Thus, there are the needs for stents with distinctive radiopacity.

WIPO Pat. Application WO01/72349 published for Pacetti et al. describes radiopaque stents formed by chemical etching, laser machining, conventional machining, electronic discharge machining, ion milling, slurry jet, or electron beam treatment or combination of these treatments of a single metal tube, or by welding of wires, or by rolling and welding of flat stock of sheet metals.

U.S. Pat. No. 6,503,271 as mentioned above describes a stent having marker tabs formed from a micro-alloyed combination of materials for visualization in a vessel. The marker tab is attached to the end of a stent after the stent is made from a metal sheet stock.

However, optimization of the radiopacity in stents is still hampered by the conventional stent manufacturing of using metal wire, sheet, or tube. The workhorse, i.e., stainless steel, in the conventional stent industry tends to cause distortion of the radiopacity of the cell near the stent. Metal alloys with superior radiopacity and other mechanical properties are underutilized because they are unsuitable for wire drawing or tube forming. Therefore, there are the needs for new manufacturing methods to broaden the options for optimizing the stent radiopacity and/or for streamlining the manufacturing steps to produce those stents. It would be even more beneficial if the new manufacturing methods could make the radiopacity features intrinsic part of the stent itself.

Stenting is an invasive procedure that can cause natural but undesirable body reaction. For example, a localized re-narrowing (i.e., restenosis) of the lumen may occur over a few months after the implantation. Inflammation of the tissue, as it could be one of the causes for restenosis, is likely to occur immediately after the implantation and may also continue for a few weeks. Therapeutic agents are thus commonly incorporated with the stenting procedure to ease such undesirable body reaction. Conventional wisdom has adopted the approaches to apply the agents on the surface of the stents or to attach the therapeutic films to the stents.

U.S. Pat. No. 5,571,166 issued to Dinh et al. discloses a method for affixing, e.g., by immersing or by spraying, the biological agents to the surface of the stents. The same U.S. patent also references the international patent applications WO 91/12779 and WO 90/13332, which disclose other methods of providing therapeutic substances to the vascular wall by means of stents.

U.S. Pat. No. 5,651,174 as mentioned above also discloses a method for making stent having a polymeric film with drug-containing microcapsules. The therapeutic film is claimed to be capable of flexing or stretching to preserve the radial expandability and axial flexibility of the implanted stent.

U.S. Pat. No. 6,361,819 to Tedeschi et al. describes a coating method to provide covalent linking of biopolymers to a substrate of medical device. The coating may be applied in multiple layers.

However, therapeutic agents are inherently fragile and thus susceptible of damage from handling. Even though efforts have been made to enhance the adhesion or to improve the mechanical properties of the polymer binders or the polymer protective layers, polymers are inherently vulnerable of damages in the absence of mechanical protection. Besides, the controls of the quantity and the elusion rate of the agent are still difficult when the agents are delivered in the form of coatings or films. Furthermore, certain high concentrations of the therapeutic agents are just unachievable due to the low solubility of the agents or the weak adhesion as a result of thick polymeric coating. Thus, there have been efforts to use additional mechanical mean of protection and elution control. For example, U.S. Pat. No. 6,206,915, as described above, discloses a stent storing the therapeutic drug in a space separated by an inner member and an outer member. However, such configuration requires more metal surface and metal mass, and thus tends to increase the rigidity and reduce the deliverability of the stent. Therefore, there are the needs for alternative manufacturing methods to produce agent-storing stents that can control the elution rates of the agents and better protect the agents, also not to compromise other properties of the stents.

### SUMMARY OF THE INVENTION

The present invention relates to articles in stent, segment of stent, and modulated stent, and also relates to methods of making those articles. The modulated stent is constructed with multiple stents or segments, which may be mixed and matched to provide various enhancements (including, but not limited to, for medical, mechanical, or delivery purpose) in the intraluminal treatments. The stents or segments are produced by metal injection molding (“MIM”), which are distinctive from the conventional manufacturing methods of using wires, tubes, or sheet stocks. Modulation processes in this invention, in conjunction with the MIM, can improve the manufacturability and ultimately reduce the costs of the stents, and provide design features that are impossible or impractical under the conventional stent manufacturing.

One aspect of the invention is directed to a stent or a segment of a stent having navigation pads, which are integrally coupled with the struts. The navigation pads exhibit distinctive patterns, i.e., radiopacity, when viewed under a diagnostic imaging technology (e.g., x-ray machine, fluoroscopy, CR scan, MRI) during the implantation of the stent. The pattern and location of the radiopacity pads can be optimized by the present method inventions.

Another aspect of the invention is directed to a stent or a segment of a stent having capabilities of storing, protecting, and delivering biological agents. The features in the present invention are integrally coupled with the main mechanical structure—metal struts. As a result, the biological agents are protected by the structure of struts, which is advantageous over the approach of using coating or strip in the conventional drug-delivery stents. Materials, designs, orientations, sizes, and mechanical properties of the struts can be tailored to serve various applications of the stents. Quantities, sizes, and locations of the reservoirs can be structured to accommodate the types, dosages, and applications of the biological agents. One embodiment of this aspect is to mold the reservoirs into the struts. The molded reservoirs thus serve dual functions, i.e., storing the biological agents and also supporting the structure of the stents. Another embodiment is to produce a porous surface on the metal struts by ways of metal powder technology and heat treatments. The depths of the pores on the porous surface can be enhanced with the etching process in conjunction with the metal powder technology.

Yet another aspect of the invention is to provide segments of a stent having interlocking pads, which are integrally coupled with the struts. The interlocking pads are used for fastening a segment of a stent to another segment. On one hand, the interlocking pads can secure the interconnection between the stent segments. On another hand, the interlocking pads can still allow bending or flexing at the interlocking joints in such way that the modulated stents can conform to the tortuous shape of the lumens, partly for ease of deployment.

Yet another aspect of the invention is to provide a stent or a segment of a stent having fastening pads, which are integrally coupled with the struts. The fastening pads are used for attaching biological membranes to the stent. The designs and location of the fastening pads can be tailored to match up with the types and the applications of the attached biological membranes.

Still another aspect of the invention is directed to a modulated stent, which is constructed by fastening together one or more embodiments (and other equivalents) as described in this invention. The modulated stent is constructed for serving multiple purposes of the stent.

A further aspect of the present invention is to provide a method for manufacturing metal stents or stent segments. The method includes one or more steps of injection molding, powder metallurgy, and other conventional metal fabrication processes. In addition, the steps of modulation are also provided to fasten several stents or segments of stents together in a cost effective and/or an operator friendly fashion.

It is further aspect of the present invention to provide choice of materials for manufacturing the stents, wherein the properties of the materials may be modified or optimized through the steps of metal injection molding and subsequent heat treatment processes. A stent or a modulated stent can have various materials or material properties at different segments of the stent.

### BRIEF DESCRIPTION OF THE DRAWINGS

FIG. 1 is a prospective view of a stent, illustrating the scaffold structure of a stent with a mono-pattern strut design.

FIG. 2 is a prospective view of another stent, including a scaffold structure similar to the structure as shown in FIG. 1 and a membrane of supporting structure.

FIG. 3 is a plan view of a modulated stent illustrating a combined embodiment of the present article invention.

FIG. 4 is an enlarged plan view of the segment 101 of FIG. 3, showing a stent or a stent segment with the navigation pads.

FIG. 5 is an enlarged plan view of the segments 102 of FIG. 3, showing a stent or a stent segment with the drug-storing reservoirs.

FIGS. 5A and 5B are sectional views of FIG. 5, showing two alternative drug-storing reservoirs.

FIG. 6 is an enlarged plan view of the segment 103 of FIG. 3, showing a stent or a stent segment with another configurations of the drug-storing reservoir.

FIG. 7 is an enlarged plan view of the segments 104 and 105 of FIG. 3, showing two stents or stent segments being fastened together by interlocking pads.

FIG. 7A is a plan view showing two stents that are fastened together by another configuration of interlocking pads.

FIG. 8 is an enlarged plan view of the segment 106 of FIG. 3, showing a stent or a stent segment with the fastening pads.

FIGS. 9 and 9A are photographs of the sectional view of a strut, showing an embodiment of porous surfaces with interconnected subsurface channels.

FIG. 10A is a prospective view of a molded and sintered part made in accordance with the present method invention, showing that the center portion of the supporting structure in a molded solid part is being removed.

FIG. 10B is a prospective view of a molded and sintered part made in accordance with the present method invention, showing that a part may be molded without the center portion of the supporting structure (in comparison with FIG. 10A).

FIG. 10C is a prospective view of a molded and sintered part with partial cut-off, showing another configuration of strut component made in accordance with the present method invention.

FIG. 11 is a prospective view of a modulated stent with three stent segments, showing that the supporting structure has been removed.

FIG. 12 is a prospective view of a modulated stent similar to FIG. 11 except that a thin layer of supporting structure is kept.

FIG. 13 is a prospective view, illustrating a step of stent modulating, where four molded stents are loaded and aligned side-by-side on a mandrel, and some adjacent struts are fastened together.

### DETAILED DESCRIPTION OF THE INVENTION

### Definitions

The term “biocompatible” or “biocompatibility” refers to the effects of materials on cells and tissues upon contact or implantation. Biocompatible materials are materials that cause no or minimal adverse effects on cells and tissues upon contact or implantation.

The term “biological agent” refers to drugs, medicines, cell replicates for medical or gene therapy at the implantation sites or otherwise chemical compounds (organic or inorganic) for property enhancement of the stents. The term “drug” is often used in place of “biological agent” in this application.

The term “elution” refers to the release process of the biological agents from the reservoirs of the stents to the tissue at or near the implantation sites during or after the implantation procedures. Elution of the biological agents is generally carried out by the body fluid.

The term “integrally coupled” refers to the formation or connection of two or more elements in an embodiment of this invention via the process of metal injection molding. The transition zone between two “integrally coupled” elements may be visually undistinguishable.

The term “segments of a stent” or other similar terms referring segments in a stent are not restricted to a component or a portion of a stent. Rather, the terms are used when such descriptions could be helpful to describe the present inventions. A “segment of a stent” can be a fully functional stent by itself from the clinical standpoint.

### DETAILED DESCRIPTION OF THE INVENTION

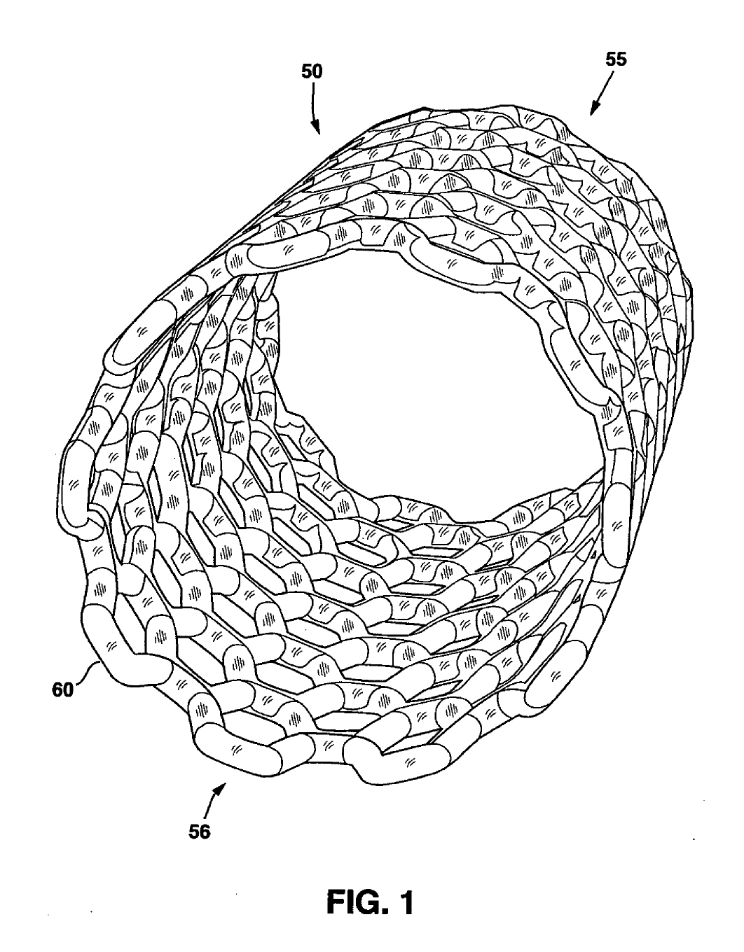

FIG. 1 illustrates the structure of a stent. The scaffold structure 50 is formed with a plurality of metal struts 60. Typically, conventional stent made of metal wires or sheets is a mono-pattern design (meaning that the pattern of the struts 60 would repeat itself throughout the stent), which is similar to the stent as illustrated in FIG. 1. The scaffold 50 conventionally is in near-round tubular shape as shown and has two open ends 55 and 56.

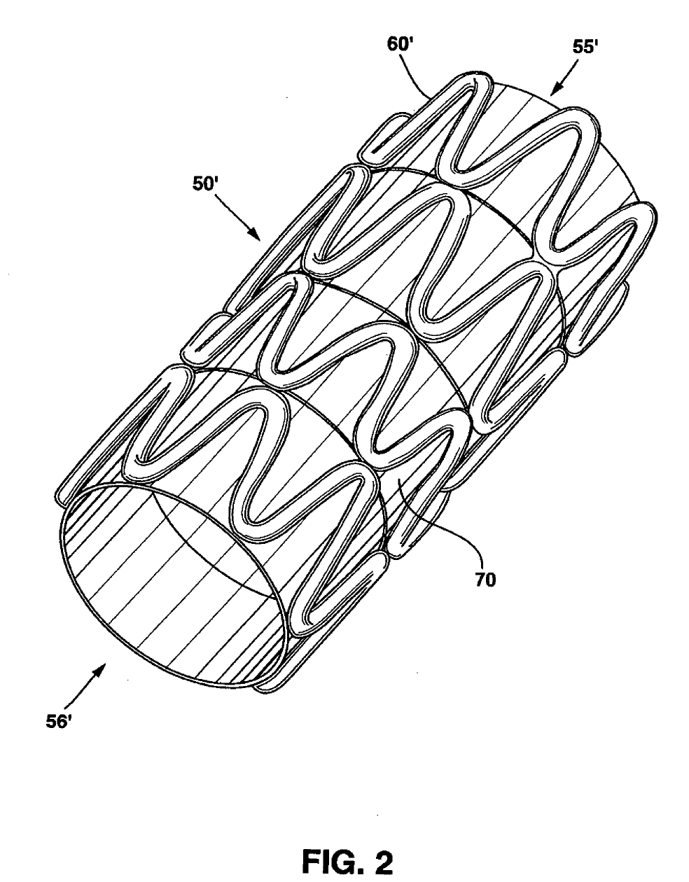

One embodiment of the present invention can be also a mono-pattern as shown in FIG. 2. The scaffold 50′ is formed with a series of struts 60′. It can also have two open ends 55′ and 56′. In addition, as will be described in detail later, it also can have a membrane of supporting structure 70.

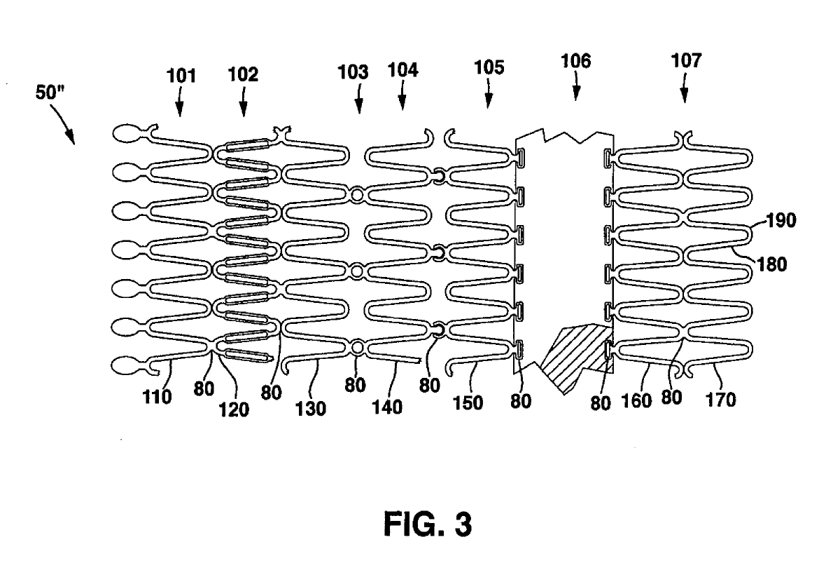

FIG. 3 illustrates a portion of one embodiment of a modulated stent in the present invention. The scaffold 50″ has a multiple segments 101, 102, 103, 104, 105, 106, and 107, connecting in series at various joints 80. The sequence of the segments 101, 102, 103, 104, 105, 106, and 107 in the scaffold 50″ does not have to be exact as shown in FIG. 3. Nor the quantities of each segment 101, 102, 103, 104, 105, 106, 107 are limited to the one as shown in FIG. 3. In other words, a modulated scaffold 50″ can have unrestricted sequences and unrestricted numbers (i.e., including a quantity of zero) of the segments 101, 102, 103, 104, 105, 106, 107, one strut segment connecting to another at the joints 80. Likewise, one segment in a modulated stent can also be a portion of another segment in the same stent. For examples, as shown in FIG. 3, segment 104 is the right-hand portion of segment 103, and segment 106 includes segment 105 and the left-hand portion of segment 107.

In comparison, a conventional metal stent (i.e., the stent made from wires, tubes, or sheet stocks) generally has mono-pattern design (as shown in FIG. 1), i.e., unlike the visually distinguishable segments as the segments 101, 102, 103, 104, 105, 106, 107. The present method inventions, as described in detail later, offer cost-effective approaches for manufacturing the modulated stent as described in FIG. 3. Conceivably, a stent with mono-pattern design is also within the scope of the present invention (i.e., the segments 101, 102, 103, 104, 105, 106, and 107 could be all visually identical).

The scaffold 50″ has a shape, including, but not limited to, a near-round tubular shape as shown in FIG. 1 or 2 (i.e., scaffold 50 and scaffold 50′ respectively). The industry today seems to have accepted the near-round tubular shape as a standard. Such shape appears to have overall acceptable levels in deliverability (i.e., ease of maneuvering through the tortuous pathway), flexibility (i.e., capability of conforming the shape of the implantation site), and capability of scaffolding (i.e., capability of withstanding the radial pressure from the lumen or capability of reducing the risk of tissue prolapse of the body cavity) of the stent, as well as in minimizing acute effects (e.g., inflammation) to the lumen as a result of the implantation. Nevertheless, the popularity of the near-round tubular shape might be merely the result of lacking alternative manufacturing methods beyond the conventional techniques of using wires or tubes. In accordance to the present method inventions (to be described in detail below), the scaffold 50″ can no longer be limited to the conventional near-round tubular shape.

The ends (they are not shown in FIG. 3 because FIG. 3 is a plan view of a portion of the modulated stent; however, the locations of the ends can be understood by referring to the two ends as illustrated in FIGS. 1 and 2, i.e., 55 and 56 in FIGS. 1 and 55′ and 56′ in FIG. 2) of the scaffold 50″ are typically open-ended. The open-ends design appears to be the present industrial standard, seemingly such design has its advantage in deployment (e.g., using balloon catheter as the deployment tool) and minimizing obstruction of flow. Nevertheless, the popularity of the open-ends design might be merely the result of lacking alternative manufacturing methods beyond the conventional techniques of using wires or tubes. The present method inventions would allow stent manufacturers to design various configurations for the ends of a stent, including, but not limited to the configuration as illustrated in FIG. 1 or 2 (i.e., the end 55, 56, 55′, or 56′).

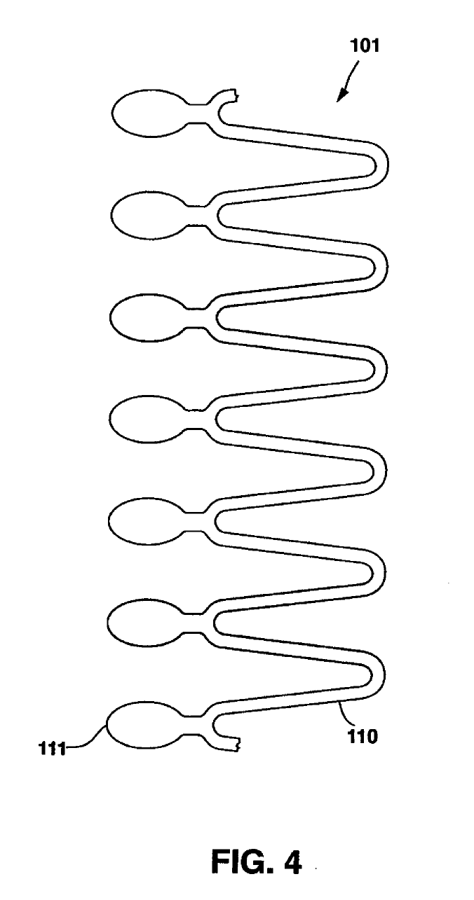

The segments 101, 102, 103, 104, 105, 106, and 107 each can have varieties of pattern design, for examples: struts 110, 120, 130, 140, 150, 160, and 170 respectively. Presently, longitudinal struts 180 and looped struts 190 appear to be two commonly adapted strut designs in the industry. As mentioned above, there have been efforts to arrange the longitudinal struts 180 and the looped struts 190 to mitigate the tendency of twisting or whipping of the stent structure made from wires, tubes, or sheet metals (e.g., in U.S. Pat. No. 6,503,271). The present stent inventions are made by metal injection molding (“MIM”) process, which can avoid some contributing factors of causing twisting or whipping (e.g., cold works in wire drawing and tube forming, sharp corners from laser cutting). As a result, the present inventions can allow other strut designs, e.g., navigation pads 111, drug-storing reservoirs 121 and 131, interlocking pads 141 and 151, and fastening pads 161, which are discussed in detail below and in FIGS. 4-8. The quantities and locations of the longitudinal struts 180, the looped struts 190, or other strut pattern designs (e.g., navigation pads 111, drug-storing reservoirs 121 and 131, interlocking pads 141 and 151, and fastening pads 161) can be determined and optimized with the considerations, including, but not limited to: the site of implantation (e.g., coronary vessel, bile duct, kidney vessel, rectum, or colon), the method of delivering the stent (e.g., delivery catheter, balloon catheter), the material of the stent (e.g., stainless steel, tantalum, nitinol, cobalt-based alloy), and other particular needs (e.g., capability in drug-storing, distinctive radiopacity).

The segments 101, 102, 103, 104, 105, 106, and 107 can be made from any biocompatible metal alloys or metal composites that are suitable for MIM process in accordance to the present method invention. Alloys and composites of titanium, 316 SS, and MP35N are some examples of the suitable candidates. It can be expected that the choices of material for the segments 101, 102, 103, 104, 105, 106, and 107 are yet to evolve while the MIM technology continues progressing. The metal alloy or metal composite of each segment 101, 102, 103, 104, 105, 106, and 107 can be different or the same. Each of the segments 101, 102, 103, 104, 105, 106, and 107 can be individually made in accordance to the present method inventions. The mechanical properties of each segment 101, 102, 103, 104, 105, 106, and 107 can also be modified or enhanced by heat treatment processes. Therefore, the present invention can allow the manufacturers ample of choices to engineer the modulated stent to fit the clinical needs.

One embodiment (FIG. 4) in this invention is for assisting stent deployment. Physicians generally prefer stents with distinctive radiopacity when viewed under a diagnostic imaging technology (e.g., x-ray, fluoroscope, CT scan, MRI) for precise placement and lesion assessment. FIG. 4 is an enlarged plan view of the segment 101 of FIG. 3. The navigation pads 111, exhibiting distinctive radiopacity, are integrally coupled to the struts 110. The distinctive characteristic in radiopacity of the navigation pads 111 can be achieved by designing the navigation pads 111 into particular shapes or patterns or using particular materials. Materials with distinctive radiopacity, e.g., titanium alloys and their composites, are some preferred materials for integral coupling to the struts 110 in accordance to the present method inventions. These preferred materials have been underutilized in manufacturing the conventional stents due to incompatibility for wire drawing or tube forming.

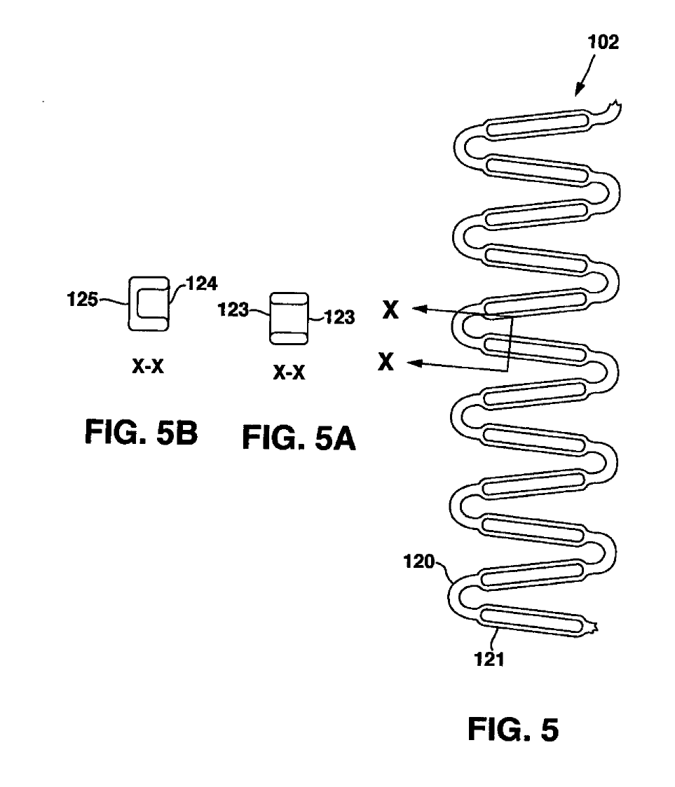

FIG. 5 is an enlarged plan view of the segment 102 of FIG. 3. The reservoirs 121, for storing and delivering biological agents, are integrally coupled to the struts 120. Biological agents (“agents”) are stored in the reservoirs 121 before the implantation. The agents can be a drug, designed to inhibit smooth muscle cell proliferation—believed to be a key contributor to restenosis or the reclogging of arteries, or can be a steroid drug to ease the inflammation of the muscle cell at the implantation site, or can be cell replicates for gene therapy. The agents can be applied to the reservoirs by injection or dispensing (in the form of solid or solution), dipping (more likely in solution form in a solvent or a polymeric liquid), or other suitable methods. The quantities of the agents can be controlled by instrumentation (e.g., injection volume control) or by the size of the reservoir 121 (e.g., certain sizes of the reservoir 121 can cause capillary effect to fill up the agents in a dipping operation). Wiping or air blowing can be used to remove excessive agents. Vacuuming can be used to remove trapped air in the solution. The solvent can be dried and the polymeric liquid can be cured with any conventional processes. After implantation of the stent, the agents are eluted from the reservoir 121 to treat the tissue surrounding or near the stent. The reservoir 121 can have different configurations, in respect to its size and shape, to match up with the types of the agents, the types of carrier for the agents, the intended treatment of using the agents, or the location of the implantation.

FIGS. 5A and 5B, as the sectional views along the line X-X in FIG. 5, illustrating two examples of the reservoirs 121. The reservoirs 121 can have two open ends 122 and 123 (FIG. 5A), or one open end 124 and one close end 125 (FIG. 5B). Coatings can be applied to cover the open end 122, 123, or 124 after the agents are applied to the reservoirs 121 to further protect or preserve the agents, or to regulate the elution of the agents from the reservoirs 121. Dissolvable coatings can be used so that a large quantity of agents can be released quickly upon implantation.

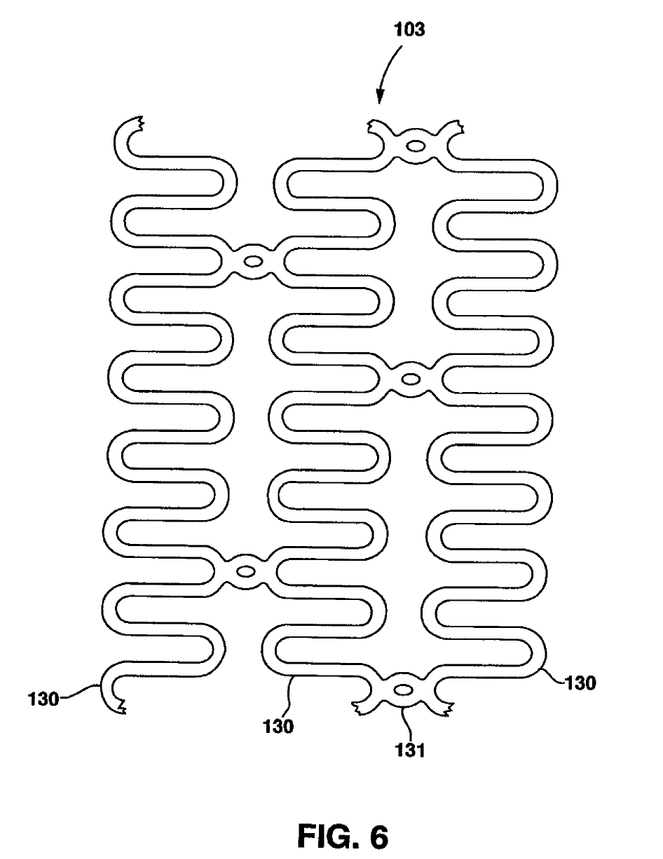

FIG. 6 is an enlarged plan view of the segments 103 of FIG. 3. The reservoirs 131, for storing and delivering biological agents, are integrally coupled to the struts 130. The specifications as described above for FIG. 5 are also largely applicable for FIG. 6. In addition, the reservoirs 131 in this embodiment also function as the connections between two segments of the struts 130. Similar to the reservoirs 121 (FIG. 5), the reservoir 131 can also have two open ends (as shown in FIG. 5A) or one open end and one closed open (as shown in FIG. 5B). Coating can be applied to cover the open ends to further protect or preserve the agents, or to regulate the elution of the agents from the reservoirs 131.

The drug-storing reservoirs 121 (FIG. 5) and 131 (FIG. 6) can also be used to benefit the mechanical structure of the segments 102 and 103 respectively. For examples, the reservoirs 121 (FIG. 5) or the reservoirs 131 (FIG. 6) can be so designed to integrally coupling with the struts 120 (FIG. 5) and the struts 130 (FIG. 6) respectively to improve the radial strength and/or minimize recoil of the segments 102 or 103. Each of the reservoirs 121 (FIG. 5) and 131 (FIG. 6) is designed to become an essential part of the structure of the struts 120 (FIG. 5) and 130 (FIG. 6) respectively.

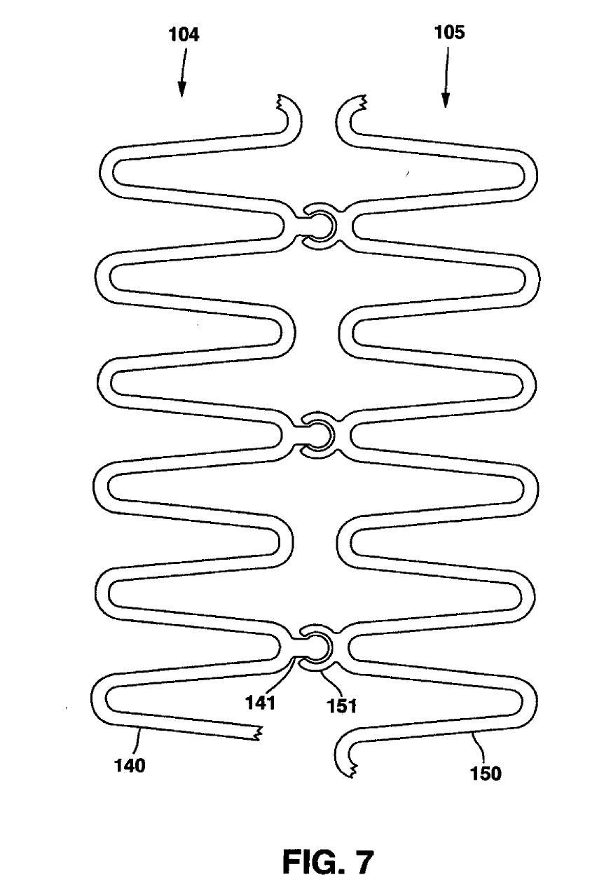

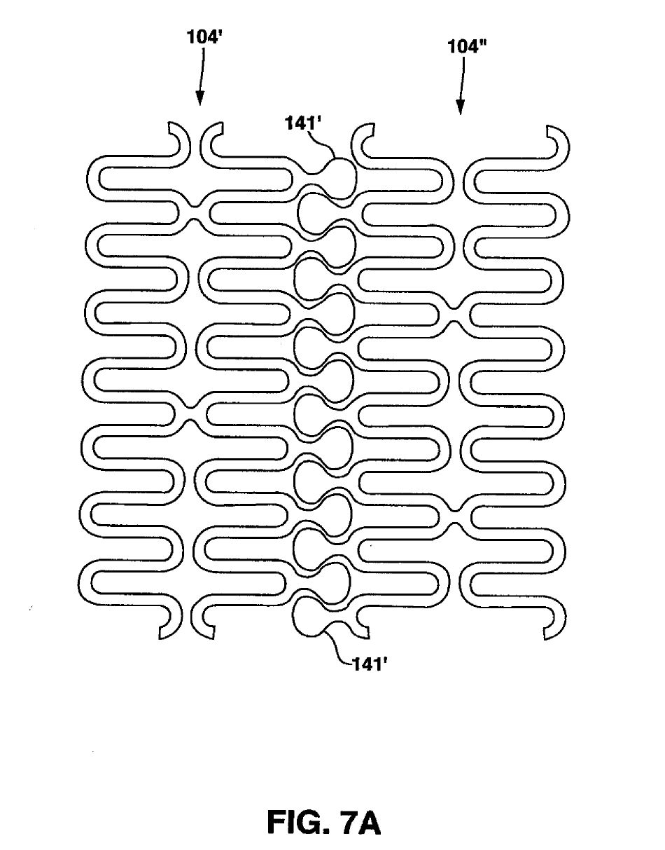

FIG. 7 is an enlarged plan view of the segments 104 and 105 of FIG. 3. The interlocking pads 141 and 151 are integrally coupled to the periphery of the struts 140 and 150 respectively. Even though the strut 140 and the strut 150 are visually alike as shown in FIG. 7, they can have different configurations. The interlocking pads 141 and 151 connect the struts 140 and 150 together.

FIG. 7A illustrates another example of the interlocking invention: two segments 104′ and 104″ are connected by the paired the interlocking pads 141′. The embodiments in the FIGS. 7 and 7A illustrate two designs, of which the paired interlocking pads 141 and 151 (FIG. 7) or the paired interlocking pads 141′ and 141′ (FIG. 7A) can restrict longitudinal movement but also allow bending or rotation between the two connected segments. Several stent segments can be connected together by the paired interlocking pads 141/151 or the paired pads 141′/141′ to maximizing scaffolding and lesion coverage.

In FIG. 7, the mating interlocking pads 141 and 151 can be designed to snap fit. More specifically, the outside diameter of the interlocking pads 141 is slightly larger than the inner diameter of the interlocking pads 151. The ball-shaped interlocking pad 141 is compressed-fitted into the donut-shaped interlocking pads 151. The friction between the two mating interlocking pads 141 and 151 in FIG. 7 thus can keep two segments 104 and 105 fastened together. It is optional that the friction between the two mating pads 141 and 151 in FIG. 7 can still allow the rotating movement between the two segments 104 and 105. The ability of the rotation movement can enhance the conformability of the stent to the tortuous implantation site but not compromise the ability of vessel wall support. Typically, the interlocked segments 104/105 as shown in FIG. 7 are interlocked together prior to the deployment of the stents.

The interconnecting mechanisms between the paired 141′/141′ (FIG. 7A) are similar to that of the paired 141/151 (FIG. 7). In other words, the designer can choose a variety of clearances between the paired pads 141′/141′, i.e., more clearance would allow easier rotating or bending between two connected segments 104′ and 104″. Conceivably, the physician may be able to interlock the two segments 104′ and 104″ inside the lumen of a body after both segments are deployed individually to the implantation site.

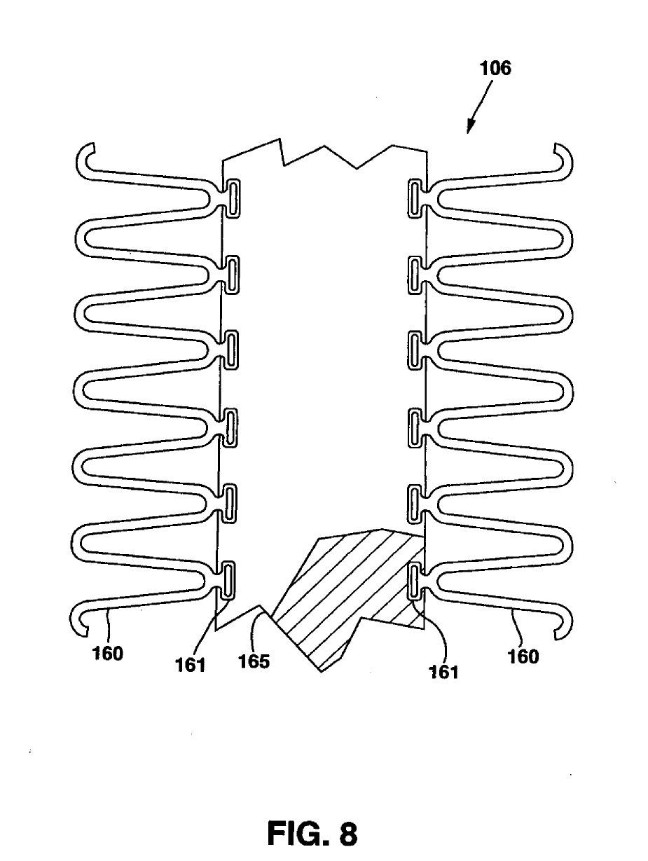

FIG. 8 is an enlarged plan view of the segment 106 of FIG. 3. The fastening pads 161 are integrally coupled to the periphery of the struts 160. The fastening pads 161 are used for attaching the membrane 165, which can carry biological agents such as drugs, genes, or nutrients. The membrane 165 can be attached to the fastening pads 161 by any traditional methods, including, but not limited to: adhesive bonding, pressing, melting, suturing, or combination.

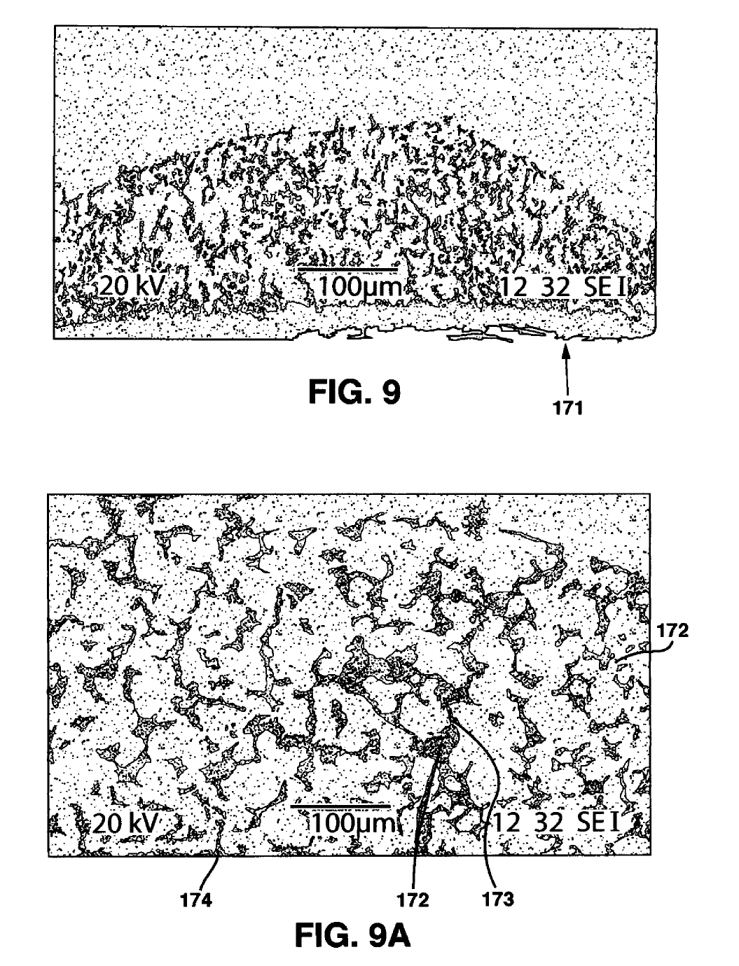

FIG. 9 is a photographic sectional view the struts 170 of FIG. 3. FIG. 9A is an enlarged view of a portion of FIG. 9, showing the pores 172 in various sizes and shapes, and some of the pores 172 are interconnected with the channels 173. The porous surface 171 are made in accordance to the method inventions, which will be described in detail below. The struts 170 having porous surfaces 171 can store and deliver biological agents. The agents are stored in the pores 172 and the channels 173 before the implantation. The agents can be a drug, designed to inhibit smooth muscle cell proliferation—believed to be a key contributor to restenosis or the reclogging of arteries, or can be a steroid drug to ease the inflammation of the tissue cell at the implantation site, or can be cell replicates for gene therapy. After implantation, the agents are eluted from the pores 172 and the channels 173 to treat the tissue surrounding or near the stent. The shape and size of the pores 172 and the channels 173 can be engineered in accordance to the present method inventions (e.g., applying heat treating process, altering metal sizes and powder/binder ratio, adjusting sintering temperature and pressure), which will be described in detail later. The length of the open space across the pores 172, as shown in FIGS. 9 and 9A, ranging from less than a microns to about 20 microns. However, larger sizes, such as a few hundreds of microns can also be produced in accordance to the present method inventions (e.g., etching process), which will be described in detail later. The outward channels 174, connecting the pores 172 and the surface of strut 170, can regulate the elution rate of the agents. Additional coating can be applied to the surface of the strut 170 to protect or preserve the agents in the pores 172 or the channels 173 and 174, or to regulate the elution of the agents.

The porous surfaces 171 can also promote cell in-growth for enhanced mechanical fixation to the implantation site. The enhanced fixation mechanism can allow, for example, the use of materials with more flexibility and/or smaller stents where the radial strength or the affixation ability might have been comprised.

The porous surface 171 can be incorporated on the surface of any segment 101, 102, 103, 104, 105, or 106. In other words, any strut 110, 120, 130, 140, 150, 160, or 170 can have the porous surface 170 for storing and delivering biological agents and/or for promoting cell in-growth. Even more, multiple types of biocompatible agents, with different quantities or elution rates, may be delivered by any of the disclosed drug-storing mechanisms (i.e., reservoirs 121, reservoirs 131, porous surface 171). The preferred materials for the present stent inventions are described in the specification for the method inventions below.

Now the specifications are directed to the methods of making the stent inventions. For ease of explanation, the method inventions are grouped into four seemingly independent, however, occasionally overlapping stages, namely: part forming, feature detailing, property enhancing, and stent modulating. For ease of viewing, only the longitudinal struts 180 and the looped struts 190 are used in the illustrative Figures for the method inventions.

The “part forming” stage is an initial step used for manufacturing each of the stent inventions. A preferred method for the part forming stage is metal injection molding technology (“MIM”), which comprises compounding, molding, de-binding, and sintering.

In compounding, metal powders are combined with a polymer or other synthetic binder, typically in a batch mixer. The mixture is then granulated (i.e., further mixed, typically in an extruder and formed the mixture into granules) to form feedstock for a molding machine. For the present article inventions, the metal powders can be selected from a group of biocompatible metals (e.g., titanium, iron, nickel, chromium, cobalt, molybdenum, aluminum, vanadium, platinum, iridium, gold, silver, palladium, tantalum, niobium, zirconium, copper, columbium, manganese, cadmium, zinc, tungsten, boron), alloys, or composites (i.e., biocompatible metals or alloys mixed with enforcement particles) for a particular stenting application. The alloys or composites can be selected to optimize, for examples, for the reasons of: manufacturability (e.g., injection molding, laser welding, heat treatment and other secondary operations), compatibility with the deployment methods (e.g., ease of transform between the unexpanded and expanded forms, flexibility for maneuvering through the tortuous pathway), capability of withstanding radial compression force from the lumen, and versatility in design (e.g., forming the above-described features such as struts, drug storing reservoirs, micro-reservoirs, interlocking pads, navigation pads, or fastening pads). The factors for selecting the binder including, but not limited to: (a) be compatible with the molding process and (b) ease to be removed (i.e., de-binding), if it is necessary, after the molding and before the sintering.

Then, the compounded powders are molded into a green part. Injection molding, compression molding, and transfer molding are among the choices for accomplishing this task. Multi-cavity molds can be used to improve the productivity and reduce the overall product costs. Multiple-shots technique may be used to form a stent with different materials or with different features. For example, the stent as shown in FIG. 4 can be produced with the following two-shot molding steps: (1) mold the main structure of struts 110 with a high strength metal material; then (2) mold a layer or a bulk of high-radiopacity material over the main structure of struts 110 where the navigation pads 111 are needed.

As mentioned above, the round or near-round tubular shape appears to be the most commonly produced metal stents in the present industry. The diameter of a tubular stent today also is generally about the same throughout the whole stent. The popularity of such stent designs might be merely the result of lacking of alternative manufacturing methods beyond the conventional techniques of using wires or tubes. The molding technique in the present invention, however, can produce various stent shapes besides the round or near-round tubular shape.

Next, the binder is removed from the molded green part (i.e., de-binding). Depending on the types of the binders, solvents or heat process can be used to remove the binder. Removing the binder before continuing the next sintering step typically will enhance the compactness of the molded structure.

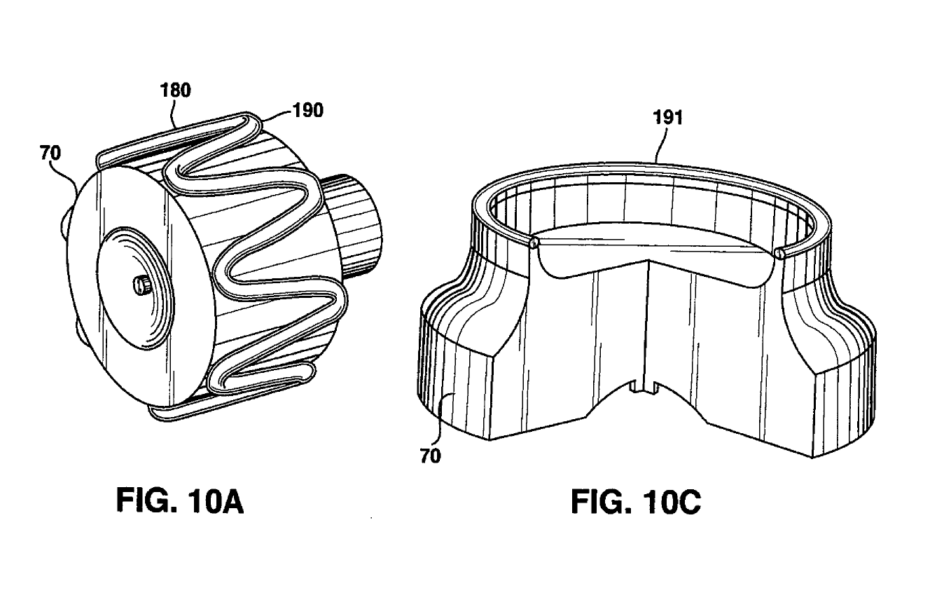

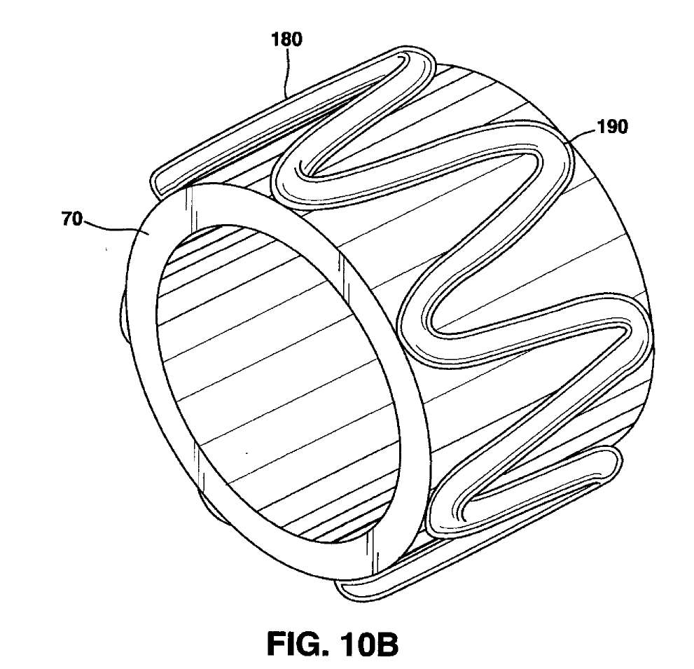

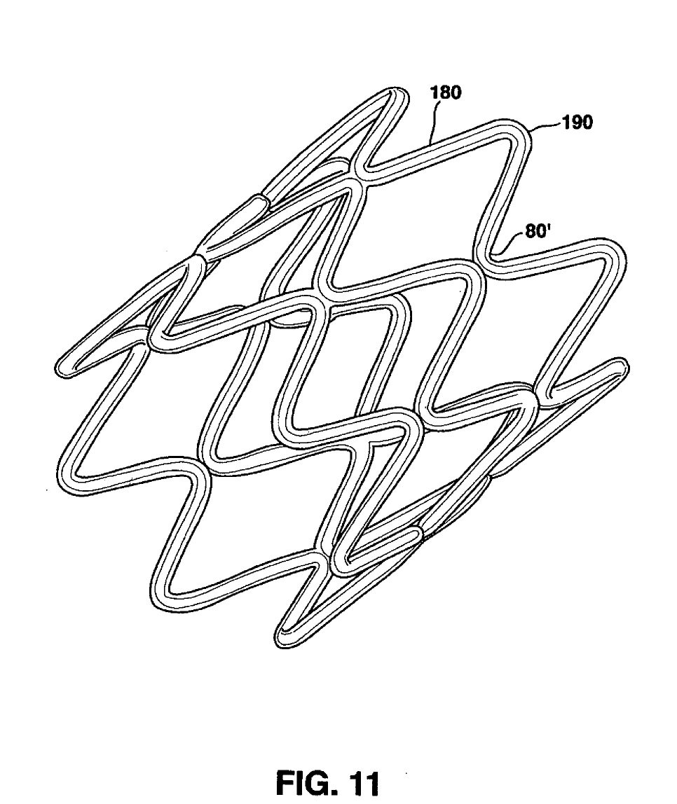

After de-binding, the structure is heated to a temperature below the melting temperature of the metal alloys to enable a re-flow of the metal alloys (i.e., sintering). Pressure can be applied during the sintering to reduce the porosity of the molded structure. FIGS. 10A, 10B, and 10C illustrate some examples of molded and sintered parts, consisting two overlapping structures: a strut structure comprising the longitudinal struts 180 and the looped struts 190 on the outer layer, and a supporting structure 70 on the inner layer. FIG. 10A illustrates that a solid part can be first molded and sintered and the center portion of the supporting structure is then removed. FIG. 10B illustrates another approach that a part can be molded and sintered without the center portion of the supporting structure. FIG. 10C illustrates another article embodiment that includes the ring structure 191 and the supporting structure 70. The ring structure 191 can be used in a particular application when it is needed. From the illustrative examples in FIGS. 10A, 10B, and 10C, those skilled in the art would be able to comprehend that the present method inventions can produce many other stent configurations.

Up to this stage, the porous surface 171 as shown in FIGS. 9 and 9A can be formed if pressure is not applied or only minimum pressure is applied during the sintering process. By alternating compounding conditions (e.g., powder/binder ratio, sizes of the powder) and sintering conditions (e.g., temperature, duration, and pressures), various configurations of the pores 172 and the channels 173 and 174 can be produced.

Further detail of MIM technology and article associated with MIM can be found in U.S. Pat. No. 6,298,901 issued to Sakamoto et al.; U.S. Pat. No. 6,428,595 issued to Hayashi et al.; and U.S. Pat. No. 6,478,842 issued to Gressel et al., which are incorporated in this application by reference.

For example, U.S. Pat. No. 6,478,842 (Gressel, et al.) discloses “a process for preparing a molded article comprising . . . admixing a feedstock comprising metal powder and binder; . . . molding the feedstock into an unsintered form; . . . removing the binder, and . . . sintering the unsintered article for a time and at a temperature sufficient to densify the molded article to at least about 95% of the theoretical density of the metal” (column 1, lines 39-48 of Gressel, et al.).

The supporting structures 70 are kept on the molded parts partly for the purposes of ease of molding, handling, or alignment in the subsequent processes. The supporting structure 70 can be removed if it is no longer needed. The removing step can be considered as a part of “feature detailing” stage as mentioned above. FIG. 11 is a prospective view illustrating three strut segments connected to each other at 80′, in a configuration when the supporting structure 70 has been completely removed. The technique for removing the supporting structure 70 can be so chosen to prevent damage to the stent structure. Laser trimming is commonly known to be an effective and precise technique of removing the metal alloys or composites.

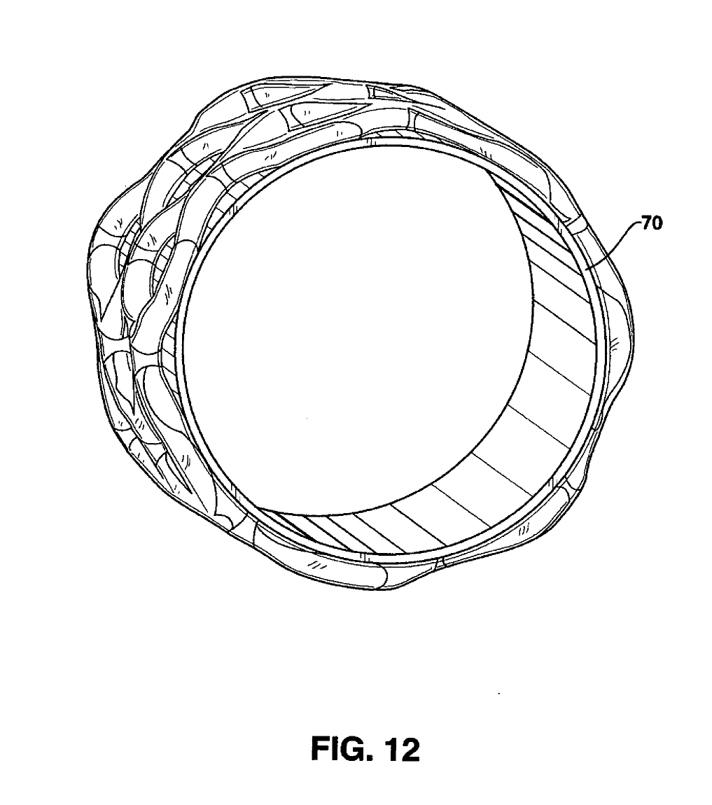

However, the boundary between the stent structure (e.g., the longitudinal struts 180 and the looped struts 190 as shown in FIG. 10B) and the supporting structure 70 sometimes is not clearly defined. That is, a portion of the supporting structure 70 may be intended to be part of the stent structure 180 and 190. As shown in FIG. 12, a thin layer of the supporting structure 70 is intentionally kept as a part of the stent structure or otherwise for ease of handling in the subsequent manufacturing processes. FIG. 2 also illustrates a modulated stent with a thin layer of supporting structure 70. In other instances, a thin layer of the supporting structure 70 can be kept to form the close-ended reservoirs 125 as shown in FIG. 5B. Yet in some other instances, a stent with a thin layer of the supporting structure 70 can withstand higher radial stress from the lumen in the implantation site.

De-burring is an optional step in the “feature detailing” stage. The stents or stent segments can be de-burred by conventional techniques such as manual polishing, electrolytic polishing, or tumbling. The de-burring can be performed either before or after the supporting structure 70 is removed. One benefit to de-burr before the removal the supporting structure 70 is that the supporting structure 70 can strengthen the structure and reduce the opportunity to damage parts in the subsequent handlings.

Yet another optional step, namely etching, can be categorized in the “feature detailing” stage in the present invention. The etching process can produce the pores 172 (FIG. 9A) of larger sizes, for example greater than 20 microns. Etching process works better when a second metal powders is added in the “part forming” stage. The second metal powders are later etched away to form the pore 172 and/or the channels 173 and 174. For example, copper and another structural metal alloy are mixed and compounded for injection molding. Once the stent is formed and sintered, the copper is then chemically or electrochemically etched away, leaving behind a network of subsurface pores 172 and channels 173 and 174. Selecting and mixing different sizes and shapes of copper can control the distribution, the sizes, and the shapes of the pores 172 and the channels 173 and 174. The duration or intensity of the etching process can control the depth toward inside the surface of the strut where the pores 172 are located. Precipitation technique or MIM can be used to make copper particles or clusters of copper with various sizes and shapes for the determination of the sizes and shapes of the pores 172, and the channels 173 and 174.

“Property enhancing” is a step to modify or to improve the properties (e.g., excellent conformability and vessel wall support, a clean optical navigation appearance, etc.) of the formed stents. Various schedules in heat treatment can be used to enhance the molded stents. Various grain sizes and mechanical properties can be achieved by the heat treatments.

The sizes and shapes of the pores 172 and the channels 173 and 174 (FIGS. 9 and 9A) can also be produced or modified in the heat treatment process. For example, first, a highly compacted stent is molded and sintered in accordance to the present method invention. The highly compacted stent would have the optimized mechanical properties. Next, metal powders, with or without the binders, are spread onto the surface of the highly compacted stent. Static electricity can be used to keep the metal powders stay on the stent surface for the subsequent process. Then, the powdered stent surface is sintered at a temperature below the melting temperature of the metal powder. The binder can be removed either before or after the sintering step. The configuration of the pore 172 and the channels 173 and 174 can be altered by using different sizes of the powders, mixing different powder/binder ratios, or applying different sintering temperatures, pressures, or durations.

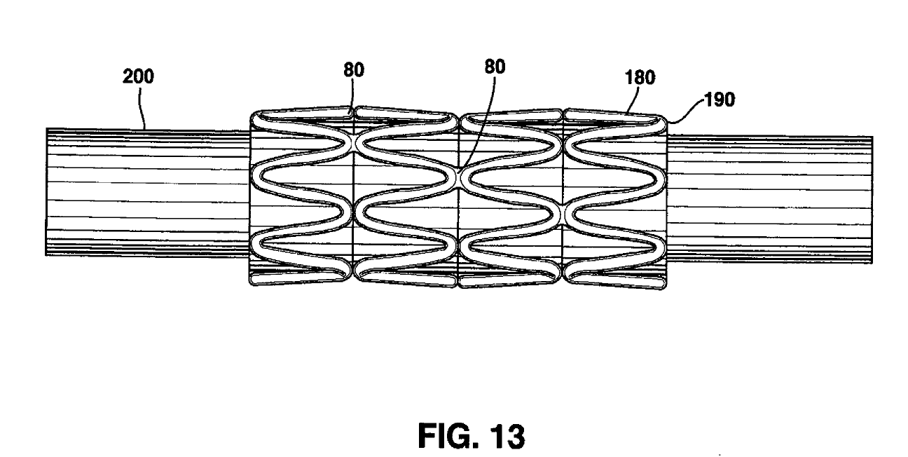

The modulated stent (FIG. 3) is made by the step of “stent modulation” of the present method invention. In FIG. 13, four molded stents with the supporting structure 70 (similar to the one shown in FIG. 10B) are loaded and aligned side-by-side on a mandrel 200. The four stents are selectively fastened (e.g., laser welding, heat fusing, ultrasonic welding, etc.) together at various joints 80 while they are loaded on the mandrel 200. The size of the mandrel 200 is so designed to have sight friction with the inside wall of the supporting structure 70. The light friction is intended to aid the ease of aligning the orientation of the stents, and to ultimately achieve high precision in alignment and high quality in fastening. The shape of the mandrel can be different from the rod shape as shown in FIG. 13. A modulated stent can be made by mix-and-match of any combinations of the molded stents as described above. Then, the mandrel 200 is removed. The supporting structure can also be removed by e.g., the laser trimming process, to form a scaffold structure similar to the modulated stent as shown in FIG. 11.

The description of the invention is intended to be illustrative. Other embodiments, modification and equivalents may be apparent to those skilled in the art without departing from its spirit.

1-20. (canceled)

21. A method for making a metal stent, comprising the steps of:
(a) compounding a mixture comprising at least one metal alloy and at least one polymer binder;
(b) molding the mixture to form a composite structure comprising a strut member and a supporting member;
(c) removing the binder from the composite structure; and
(d) sintering the composite structure to achieve at least about 95% of the theoretical density of the metal alloy.

22. The method of claim 21, further comprising removing at least a portion of the supporting member from the sintered composite structure.

23. The method as in claim 21 or 22, further comprising etching the surface of the stent.

24. The method of either of claims 21 or 22, further comprising heating the stent to alter a surface or mechanical property of the stent.

25. The method of claim 24, further comprising placing at least one metal powder on the surface of the stent before heating.

26. A method for making a modulated stent, comprising:
(a) compounding a mixture comprising at least one metal alloy and at least one polymer binder;
(b) molding the mixture to form two or more composite structures, each composite structure comprising a strut member and a supporting member;
(c) removing the binder from each of the composite structures;
(d) sintering the composite structures to achieve at least about 95% of the theoretical density of the metal alloy;
(e) aligning two or more of the composite structures on a mandrel;
(f) fastening the composite structures together to form the modulated stent; and
(g) removing the modulated stent from the mandrel.

27. The method of claim 26, further comprising removing at least a portion of the supporting member from the sintered composite structures either before the composite structures are aligned on the mandrel or after the modulated stent is removed from the mandrel.

28. The method of either of claims 26 or 27, further comprising etching the surface of the stent.

29. The method as in claims 26 or 27, further comprising heat treating the stent to alter a surface or mechanical property of the stent.

30. The method of claim 29, further comprising placing at least one metal powder on the surface of the stent before heating.
